(19) United States
(12) Patent Application Publication
(10) Pub. No.: US 2021/0369431 A1
(43) Pub. Date: Dec. 2, 2021

(54) DENTAL CARE MOUTHPIECE CLEANING SYSTEM

(71) Applicants: Frank Dreier, Weston, FL (US); Thomas Pinnau, Boca Raton, FL (US)

(72) Inventors: Frank Dreier, Weston, FL (US); Thomas Pinnau, Boca Raton, FL (US)

(21) Appl. No.: 17/346,688

(22) Filed: Jun. 14, 2021

Related U.S. Application Data
(63) Continuation of application No. PCT/US2019/066309, filed on Dec. 13, 2019.
(60) Provisional application No. 62/779,606, filed on Dec. 14, 2018.

(51) Int. Cl. A61C 17/22; A61C 17/20; A61N 5/06

(52) U.S. Cl. CPC A61C 17/228; A61C 17/20; A61N 5/0603; B06B 1/0292; A61N 2005/0606; A61N 2005/0652; A61N 2005/0661; A61N 5/0624

(57) ABSTRACT

A mouthpiece cleaning device includes a U-shaped mouthpiece skeleton that defines opposing channels to receive a user's teeth therein. The channels each have a respective teeth facing surface, and transducers are disposed along the channels at the respective teeth facing surfaces. UV LEDs are disposed along the channels at the respective teeth facing services. The UV LEDs are interspersed with the transducers.

### CROSS-REFERENCE TO RELATED APPLICATION

This application is a continuation, under 35 U.S.C. § 120, of copending international application PCT/US2019/066309, filed Dec. 13, 2019, which designated the United States; this application also claims the priority, under 35 U.S.C. § 119, of provisional patent application No. 62/779,606, filed Dec. 14, 2018; the prior applications are herewith incorporated by reference in their entirety.

### BACKGROUND OF THE INVENTION

### Field of the Invention

The invention relates to a mouthpiece that cleans a user's teeth and gums.

People have always had trouble routinely brushing and flossing. Once the plaque is allowed to form on the teeth close to the gums, over the course of just a few hours, the bacterial microflora change to more pathogenic anaerobes that can cause disease. An ideal oral care regimen needs to be easy to use, short in duration, provide good cleaning of all teeth and gum surfaces, be healthy and safe in the long term, and must fit into people's lifestyles. In the past, the requirement of a minimum of 2 minutes of brushing and 2 minutes to floss has not seen great success because of the technique sensitivity (lack of user's ability) and the amount of time required to fit into people's busy lifestyles. Wrong/Insufficient tooth care techniques lead to insufficient/uneven dental cleaning and even abrasion, if too much pressure is used during the cleaning process.

Key current oral health cleaning issues that the present invention will overcome include the following: wrong brushing habits e.g. insufficient cleaning (not all surfaces equally well), too short time (not clean enough), too much brushing pressure (causing damaging to teeth and gums), too short cleaning process (less than 2-3 minutes); and all current oral cleaning systems depend on the users' know how and capability how to properly clean all teeth (which we know is not the case for many people).

U.S. Pat. No. 7,044,73762 discloses the use of ultrasound transducers in a mouthpiece-like device for oral health care benefits. Other references disclose the use of UV light for teeth whitening for the frontal teeth.

The prior art devices generally disclose the use of ultrasound, sonic vibration and UV light separately to provide oral cleaning benefits in general terms. The prior art devices also do not supply a mouthpiece cleaning system with ultrasound, sonic vibration and UV light together, using a dentifrice toothpaste/toothgel for customized oral health benefits to retain or restore oral cleanliness and health. The prior art devices also do not use ultrasound in combination with UV lights all disposed in a mouthpiece for the front, the back and the top of all teeth to provide enhanced cleaning, antibacterial and therapeutic benefits.

Further, prior art devices clean by brushing methods work that work through abrasion, e.g. removing bacterial plaque through brushing. This has the inherent risk of abrasion, can lead to receding gum lines and sensitive teeth. The present invention benefits with touch-less cleaning with ultrasound and completely eliminates this risk—no brushing, no risk of hurting of gums, no abrasion and the complete elimination of human insufficiencies in the oral cleaning procedure.

Traditional prior art cleaning methods by brushing have the risk of causing damage to gums/teeth/braces/implants—even if the user is brushing properly. Brushing is usually not permitted after oral surgeries. There are great difficulties of cleaning behind braces because bristles do not reach and secondary cavities can develop. The present invention has touch-less deep cleaning with ultrasound waves can be used even with most sensitive gums, inflamed gums, before or after oral surgeries (e.g. pin-hole-technique, gum surgeries, implants)—because ultrasound touchless removal of inflammation causing bacteria (cavitation) and the ultrasound waves penetrating the gums providing additional health benefits.

Furthermore, traditional cleaning methods by brushing have limited personalization/customization abilities for oral cleaning (increase decrease brushing oscillation and choice of toothpaste). The present invention mouthpiece has multiple different parameter (ultrasound frequencies, ultrasound power levels, UV light at different frequencies, sonic vibration as different frequencies, range of different toothpastes. This allows customization to an individual's cleaning, health and therapeutic needs, in a single device.

Prior devices that use piezo technology have limitations in shape, power field deliveries—that the CMUT technology of the present invention overcomes. Notwithstanding, piezo chips may be used in the present invention where necessitated.

The prior art does not have the unique combination of Ultrasound, UV and Sonic Vibration and range of toothpastes disclosed in the present invention that now delivers cleaning and health benefits.

Traditional cleaning methods by brushing do surface cleaning only. They cannot reach inside tiniest micro-gaps and cannot penetrate gum tissue. The ultrasound waves generated by the present invention mouthpiece reach deep inside tiniest crevices to remove bacteria and they also penetrate the gums—micro-massaging the gums, thereby increasing the temperature and blood circulation to accelerate gum healing/regeneration.

Prior art cleaning methods by brushing rely on human skills to achieve good cleaning results. In the present invention, the mouthpiece is a no-fail, always perfect, auto-pilot cleaning method. No matter the user's skill level.

### BRIEF SUMMARY OF THE INVENTION

It is accordingly an object of the invention to provide a dental mouthpiece cleaning system, which overcomes the hereinafore-mentioned disadvantages of the heretofore-known devices of this general type and which is automated, simple and efficient to use (merely requires placement in a mouth and actuation).

With the foregoing and other objects in view there is provided, in accordance with the invention, a mouthpiece cleaning device includes a U-shaped mouthpiece skeleton that defines opposing channels to receive a user's teeth therein. The channels each have a respective teeth facing surface, and transducers are disposed along the channels at the respective teeth facing surfaces. UV LEDs are disposed along the channels at the respective teeth facing services. The UV LEDs are interspersed with the transducers.

The system according to the invention strongly reduces overall cleaning time, increases the cleaning time per each tooth surface, cleans gums and all teeth surfaces equally long and thorough, is safer (no abrasion risk) and provides therapeutic health benefits to the gums. The mouthpiece with ultrasound transducers, ultraviolet LEDs and a sonic vibrator that insertable to a mouth to clean teeth and gums by removing bacterial plaque, that cause gum inflammation, thereby accelerating the healing process and to provide other therapeutic benefits. The mouthpiece is constructed to reach all teeth and gum surfaces evenly as well as interdental spaces.

In accordance with another feature of the invention, the transducers are PMUTS or CMUTS or piezo transducers.

In accordance with another feature of the invention, a sonic vibration chip is disposed in the skeleton.

In accordance with another feature of the invention, the transducers and the LEDs are embedded in the skeleton by injection molding.

In accordance with another feature of the invention, the mouthpiece is a solid or semi-solid polymer, or gel based material, being PVC, polyethylene, PEEK, polycarbonate, polyetherimide, polysulfone, polypropylene or polyurethane.

In accordance with another feature of the invention, there is a flexible circuit board, the transducers and the LEDs are disposed on the flexible circuit board.

In accordance with another feature of the invention, a controller is connected to the flexible circuit board. The controller controls the transducers, the LEDs and the vibration unit.

In accordance with another feature of the invention, there is an overmolded layer of material softer than a material of the skeleton.

In accordance with another feature of the invention, spacers are disposed in the channels at the respective teeth facing surface. The spacers are to contact the user's teeth and maintain a minimum distance between the transducers and the teeth. The spacers are formed of the overmolded layer.

Other features which are considered as characteristic for the invention are set forth in the appended claims.

Although the invention is illustrated and described herein as embodied in a dental mouthpiece cleaning system, it is nevertheless not intended to be limited to the details shown, since various modifications and structural changes may be made therein without departing from the spirit of the invention and within the scope and range of equivalents of the claims.

Other features which are considered as characteristic for the invention are set forth in the appended claims.

Although the invention is illustrated and described herein as embodied in dental care mouthpiece cleaning system, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1 is a diagrammatic, top view of the dental mouthpiece cleaning device;

FIG. 2 is a diagrammatic rear-side view of the mouthpiece of the dental mouthpiece cleaning device;

FIG. 3 is a diagrammatic section view of the mouthpiece of the dental mouthpiece cleaning device showing the channels with tooth cleaning medium;

FIG. 3 A is a section view of the mouthpiece of the dental mouthpiece taken through a transducer;

FIG. 3B is an enlarged diagrammatic section view of the mouthpiece of the dental mouthpiece taken through a transducer to show the material layers;

FIG. 3C is a section view of the mouthpiece of the dental mouthpiece taken through an LED;

FIG. 3D is an enlarged diagrammatic section view of the mouthpiece of the dental mouthpiece taken through an LED to show the material layers;

FIG. 4 is a perspective view of the mouthpiece inserted in a user's mouth;

FIG. 5 is a view of application of tooth cleaning medium onto the mouthpiece;

FIG. 6a is a hardwire mouthpiece wiring diagram;

FIG. 6b is a wireless mouthpiece wiring diagram; and

FIG. 7 is a diagrammatic section of a flex circuit.

### DETAILED DESCRIPTION OF THE INVENTION

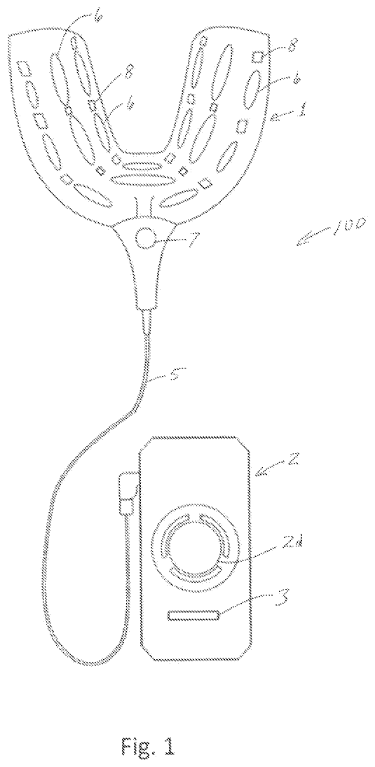

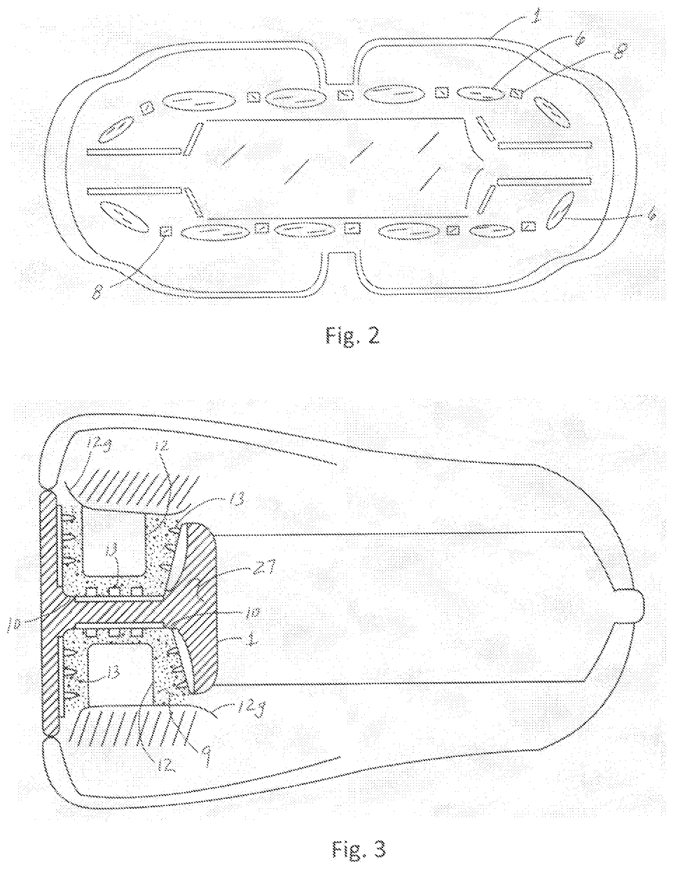

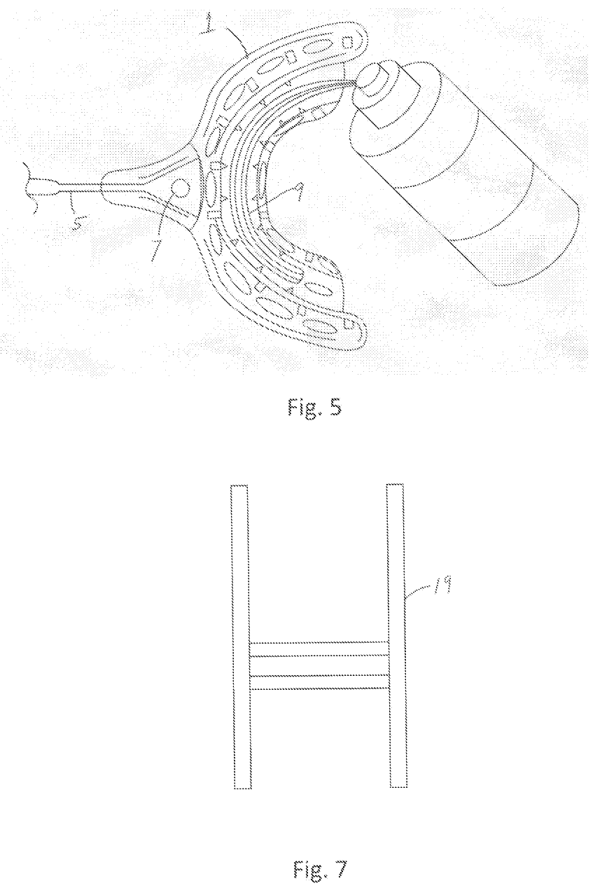

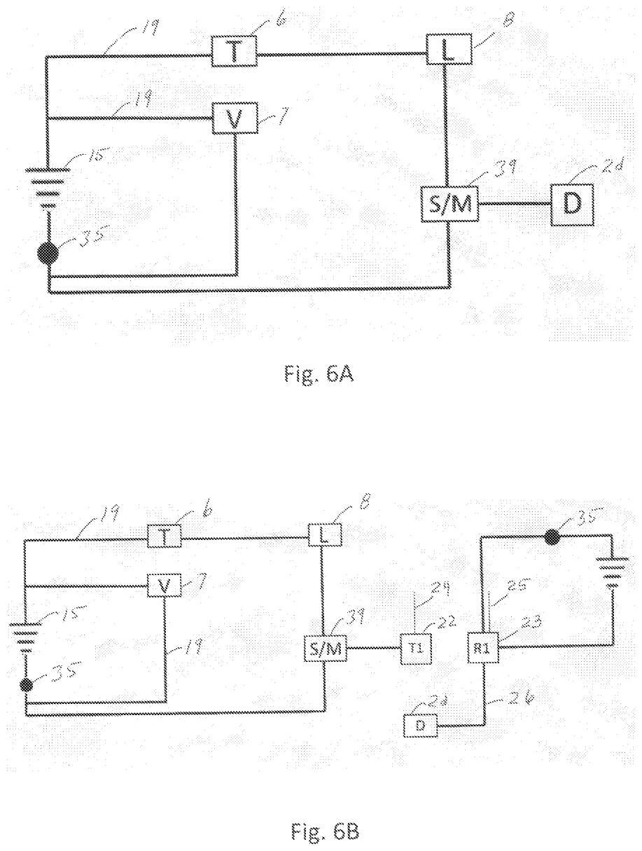

Referring now to the figures of the drawing in detail and first, particularly, to FIG. 1 thereof, there is seen a mouthpiece cleaning device 100 having a mouthpiece 1 electrically connected to a controller 2 by hardwiring. The controller 2 has a display 2d the display can be LCD, digital, or may simply be LEDs that changes colors, to show the device 100 is on and can also show the power level or cleaning program that is being run. The cleaning programs will be discussed further hereinafter. As seen from the wiring diagram in FIG. 6B, it is also possible for the controller 2 to be connected wirelessly to the electrical components of the mouthpiece 1. The controller 2 is provided with a selector/power button 3 to control the operation of the device 100.

The mouthpiece 1 is provided with ultrasonic transducers/transmitters 6, which are driven by a power source 15 such as a rechargeable battery. The transducers 6 can be provided as either PMUTs (Piezoelectric Micro-machined Ultrasonic Transducers) or CMUTs (Capacitive micro-machined ultrasonic transducers), which with respect to transducer technology have important technology advantages over conventional piezo transducers (lower operational power levels, flexible in shape, more even ultrasound transmission patterns, less space requirements). Different from conventional piezo transducers, CMUT/PMUT use a 3-dimensional mesh structures that provide a very even, continuous and equally strong ultrasound wave distribution field. In the case of the present invention, this means a very even, continuous and equally strong ultrasound wave distribution field across the entire oral orifice, which allows for a superior, even cleaning across all parts of the teeth and gum area.

The controller 2 provides power supply and sets/manages the mouthpiece's different operation modes. The controller 2 may be external (connected by cable 5 to the mouthpiece 1) or internal (integrated into the mouthpiece 1). The controller 2 includes a rechargeable battery, firmware electronics, operating buttons and indicators. The controller 2 manages the power supply, contains a rechargeable battery, electronics (boost the battery voltage by a voltage multiplier circuit to the range of 9.6 VDC to 16.0 VDC). The controller 2 may have an ON/OFF switch 3 with LED light indicators/display 2d.

The LED control lights 2d will indicate:
On/off.
Show that connected to external power supply and that charging is on.
Show that mouthpiece 1 is connected and operational.
Battery charging level indicator: Fully charged/charging status/needs charging.
Mouthpiece replacement need indicator (3 stages—green, yellow, red).
Lights will indicate wear out status of mouthpiece embedded transducers 6—and signal the need for replacement on number of usages/cycles (internal counter).

UV LED Lights 8 and sonic vibration chip 7 will indicate:
Unit is operational and all electronic elements functioning.
Start/End of cleaning time—beeping sound (and or vibration and/or UV LED lights 8) to indicate the mouthpiece 1 is operating and/or finished cleaning procedure.
Different cleaning/therapy modes (basic cleaning, intense cleaning, gum care/gum health). Modes will differ in application time, ultrasound intensity, ultrasound transducers 6 on/off operating modes (oscillation pattern) applied and the recommended tooth gel or toothpaste for each operation mode.

The controller 2 will offer several mouthpiece 1 operational settings for different health care benefits, providing a unique combination of i) ultrasound frequency, ii) modulation of power level, and iii) length of ultrasound exposure or the use of continuous vs pulsed ultrasound. The different settings can create a different stimulatory effect through the acoustic streaming of the ultrasound and the cavitation effect. Lower frequency settings can increase the level of cavitation, which can increase the effect of bacterial plaque removal and micro blood circulation, while higher frequency levels allow for lower power levels. Examples of some (but not limited to) operational setting can be:
Regular Cleaning modus (Frequency x1, Power Level y1, Lengths z1)—bacterial plaque removal
Intense Cleaning modus (Frequency x2, Power Level y2, Lengths z2)—in-depth bacterial plaque removal
Gum Health Modus (Frequency x3, Power Level y3, Lengths z3)—increase micro blood circulation, etc.
Teeth Whitening Modus (Frequency x4, Power Level y4, Lengths z4)—activate gel whitening ingredients
Settings will be determined to provide maximum health benefit efficiency for the different applications.
The above listed settings may applied equally to all ultrasound transmitters 6 of the mouthpiece—or to selected transmitters 6 only in order to promote targeted health and cleaning benefits for specific mouth areas.
Each setting corresponds to a special dentifrice gel or paste formulated to enhance the desired benefits.

The controller is connected by cable or by induction to power supply (110 Volts or via USB) to recharge the internal batteries.

The mouthpiece 1 ss U-shaped (unisex/universally shaped or available in multiple sizes) and has opposing channels 10 to reach all teeth/gum surfaces when inserted into the mouth. The mouthpiece 1 is made out of skeleton 27 of solid or semi-solid polymer, or gel based material, such as but not limited to: PVC and polyethylene, PEEK, polycarbonate, Ultem (a trademark of SABIC Global Technologies B.V. besloten vennootschap (b.v.) NETHERLANDS), PEI known as polyetherimide, polysulfone, polypropylene and polyurethane. The material will have a high transmission rate of ultrasound to facilitate a strong and even transmission of ultrasound to teeth and gums. The ultrasound transmitters 6, sonic vibration unit 7, UV lights 8, and the flex circuit board 19, are embedded into the material 27, which may also be referred to as a skeleton. The elements 6, 7 and 8, are connected by a cable to the external power supply/controller unit. In case of the wireless execution, the mouthpiece will include an internal power supply and be connected wirelessly to the external controller unit and the elements 6, 7 and 8 are connected by the flexcircuit board to the internal power supply. The mouthpiece may have an overmold of a softer semi solid based material 29, such as but not limited to polyutherene or silicone—to provide a better, more comfortable and more universally shaped mouth-fit.

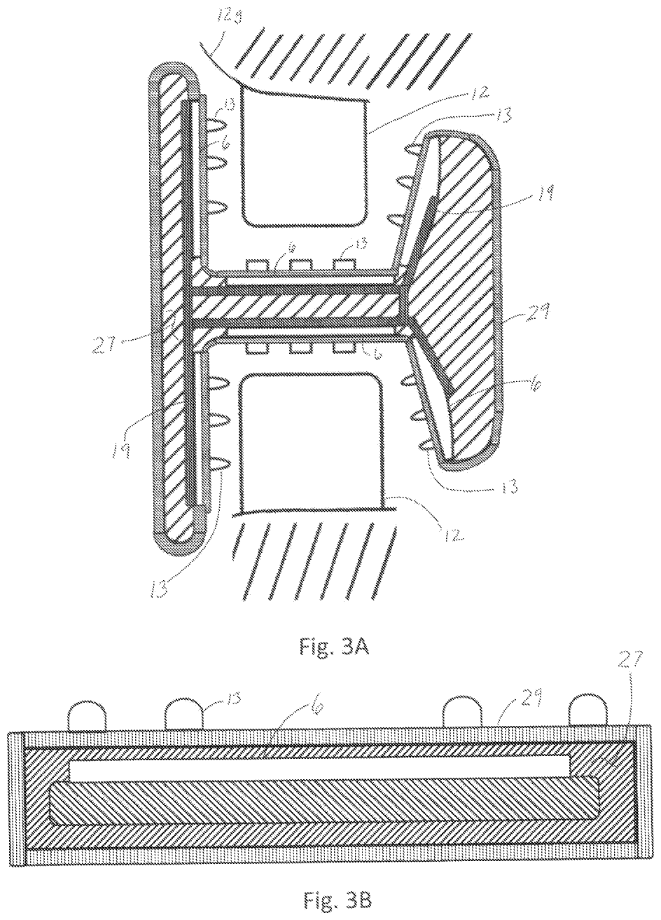

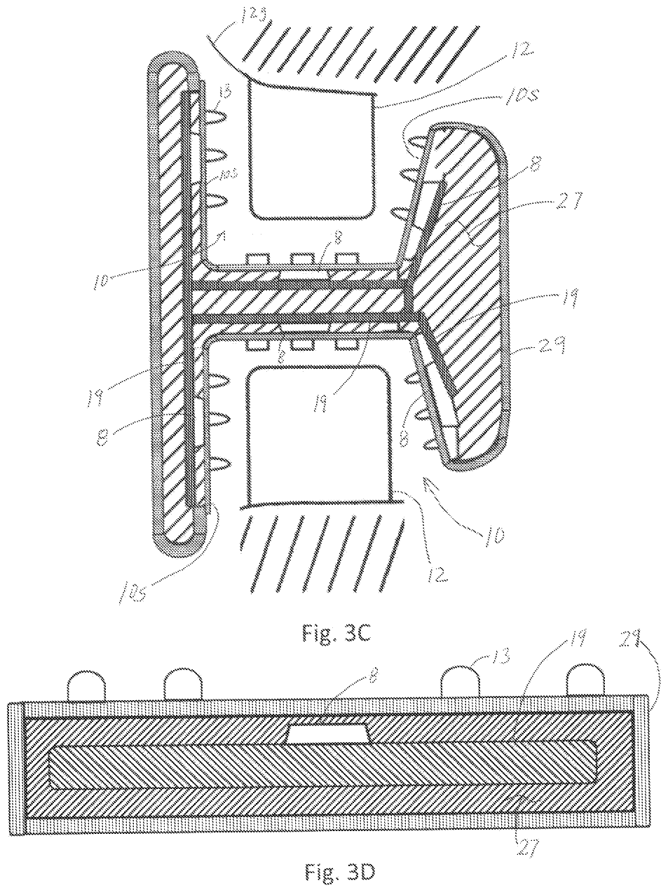

As shown in the different FIGS. 3, the mouthpiece overmold 29 may be molded to include spacers 13 for all teeth facing surfaces to protect the ultrasound transducers 6 and to maintain a minimum distance of 2 to 5 mm between each tooth surface and transducers 6 for optimum ultrasound wave delivery and for increased comfort and fit of the mouthpiece 1. The spacers 13 serve as standoffs to create a minimum void area that will be filled with tooth cleaning medium 9 and naturally occurring human saliva that acts as the medium for the ultrasound wave transmission and provides for the cleaning action (cavitation effect). The mouthpiece base construction may include short bristles, to further support the removal of loose debris/plaque (sonic vibration) and the more effective transmission of ultrasound waves.

The transducers 6 are molded inside a solid or semi-solid polymer, or gel based material 27 (as described above), or in addition Zerdine (a registered trademark of Computerized Imaging Reference Systems, Inc. Norfolk Va.) from cirsinc.com with adjustable properties speed of sound: 1480-1650 m/s (attenuation coefficient: 0.1 to 1.5 dB./cm/MHz with a linear frequency response). This material(s) will have a high transmission rate of ultrasound to facilitate a strong and even transmission of ultrasound to teeth 12 and gums 12g.

The transducers 6 will use LIPUS (low intensity high frequency ultrasound). Power and frequency levels will be between 20 kHz and 20 MHz frequency, more preferably within 750 kHz and 2 MHz frequency, at a non-attenuated intensity from 0.02 to 0.5 W/cm², more preferably within 0.035 to 0.150 W/cm². The ultrasonic cleaning effect is based on the principle of cavitation. Oscillations with frequencies above 20 kHz are called ultrasound and are no longer in the human hearing range. The frequencies below a power limit of 50 watts are absolutely safe for human body and harmless. By an ultrasonic vibrator high frequency electrical energy is converted into mechanical vibration. The high frequency vibrations in liquids result in pressure and vacuum oscillations that create micro-bubbles smallest cavities (vacuum bubbles) which collapse (implode) in rapid succession to create enormous impact energies between the detergent liquid (tooth gel and saliva) and the surface to be cleaned—referred to as the principle of cavitation. These movements (cavitation) result in the dirt on the surface to be cleaned and provides for the gentle removal of impurities. The effect of ultrasound in oral hygiene has been scientifically proven to destroy the outer shell of bacteria and germs, and that it does not cause damage to the human body at the indicated transmission frequencies and power levels. The gums 12g are gently massaged, and as harmful bacteria are removed inflammation subsides and gums 12g can start to regenerate again. In this micro-purification process natural teeth, crowns and bridges, implants and other fixed restorations, as well as the micro gaps and interdental spaces, inside tiniest spaces and micro-gaps, between implants and bone, between filling and enamel, between braces and teeth, that ordinary cleaning methods with bristles cannot reach, are cleaned thoroughly.

The number of ultrasound transducers 6 will depend on technology choice. PMUTs (Piezoelectric Micro-machined Ultrasonic Transducers) or CMUTs (Capacitive micro-machined ultrasonic transducers) will be the base choice transducer technology as they have important technology advantages (lower operational power levels, flexible in shape, more even ultrasound transmission patterns, less space requirements). Different than conventional piezo transducers, CMUT/PMUTs use 3-dimensional mesh structures that provide a very even, continuous and equally strong ultrasound wave distribution across the entire oral orifice which allows for a by far superior cleaning across all parts of the teeth and gum area.

Ultrasound frequencies, power levels and patterns will be set according to the desired cleaning and/or health care benefits and selected at/managed by the external controller unit 2.

The mouthpiece 1 will include UV light emitting LEDs 8 that are proven to have antibacterial benefits. The use of UV light in oral care is known to kill bacteria in the 470-490 mm wavelength range. As seen in FIG. 1, the LED's are disposed among/between the transducers 6 and are molded into the material 27 together with the transducers 6 and subsequently overmolded by the overmold material 29 of the mouthpiece 1. As seen in FIG. 3C, it is possible that the material 27 does not cover the outer surface of the LED 8, but terminates flush therewith. As seen in FIG. 3D it is possible that the surface of the LED directed toward the tooth is covered by a thin layer of the skeleton material 27.

A vibration chip 7 is provided in the mouthpiece 1 to generate a gentle sonic vibration. The vibration will aid the removal of loose debris/plaque, while also providing a sensory signal to indicate that the mouthpiece and its ultrasound action is in active cleaning/operating mode.

FIGS. 3A-3D show the disposition of the element and the relationship of the molded material to the different elements. FIGS. 3A and 3B show a section through the mouthpiece 1 that is taken at a position through the transducers 6. Here it is shown that the transducers 6 are disposed on the flex circuit 19 and the material of the skeleton 27 surrounds the transducers 6 and the flex circuit 19 in a shape that defines the channels 10, which define the teeth facing surfaces 1 Os at which the transducers are provided (FIG. 3B shows that the skeleton material 27 can also cover the tooth facing surface of the transducer 6). The skeleton material 27 and thus the transducers 6 are covered by the overmolded material 29, which defines the spacers 13. FIGS. 3C and 3D show a section through the mouthpiece 1 that is taken at a position through the UV LEDs 8. Here it is shown that the UV LEDs 8 are disposed on the flex circuit 19 and the skeleton material 27 surrounds the UV LEDs 8 and the flex circuit 19 in a shape that defines the channels 10, which define the teeth facing surfaces 10s at which the transducers are provided (FIG. 3D shows that the skeleton material 27 can also cover the tooth facing surface of the UV LEDs 8). The skeleton material 27 and thus the UV LEDs 8 are covered by the overmolded material 29.

The wiring options for the device 100 are shown in FIGS. 6A and 6B. As seen, the ultrasound transducers 6, the UV-light LED's 8 and the vibration chip 7 are all individually wired and controlled by a sensor/micro-processor 39 and connected to a battery 15 with a switch 35, which signals the devices 6, 7, and 8 and allows for independent sequencing, application time, order of application and benefits for varying oral health care benefits. The sensor/microprocessor 39 senses that the individual devices 6, 7, and 8 are functioning as signaled. The wiring will be low electrical capacitance, shielded and insulated, non-corrosive, high resistant to body fluids and temperature resistant. The wiring will be enabled thru high connectivity electrical flex circuit boards 19, the flexible feature allowing to bend the board to the shape of the mouthpiece 1. The transducers 6 and LED's 8 are disposed on the flex circuit boards 19 along the length thereof. For the wireless mouthpiece 100 execution wiring will include a transmitter/receiver 22 with antenna 24 and a receiver/transmitter 23 with antenna 25 which is then connected to the display 2d by hard wire 26.

The mouthpiece 1 has electric flex circuit boards to supply power to internal sides of the mouthpiece. The flex circuit boards 19 are shown in the FIGS. 3 and the elements 6, 7, 8 are disposed on the flex circuit board 19.

Besides the sonic vibration and UV light emitting LED's, other sensory signals may include control LEDs 2d and a beep sound to indicate operation mode (on/complete/off).

The mouthpiece 1 for the wireless version, includes a rechargeable battery 15 (located inside the u-shape area for the wireless connected mouthpiece version), a charging connection, controller electronics unit 29 (located in front of lips area/outside mouth) with on/off switch with LED indicator and wireless or bluetooth capability to control device via a smartphone application (integrated controller unit).

The mouthpiece 1 may add ultrasound imaging/sensing. The ultrasound transducers 6 are constructed to emit and receive ultrasound signals as used in ultrasound imaging. This feature can be used to take ultrasound images of teeth 12 and gums 12g, using software to analyze and detect bacterial plaque, gum inflammation and the overall health status of the mouth. Based on this analysis and via the controller 2 and an application, a mouth piece cleaning mode recommendation can be provided and the oral cleaning and oral health status of the user can be tracked.

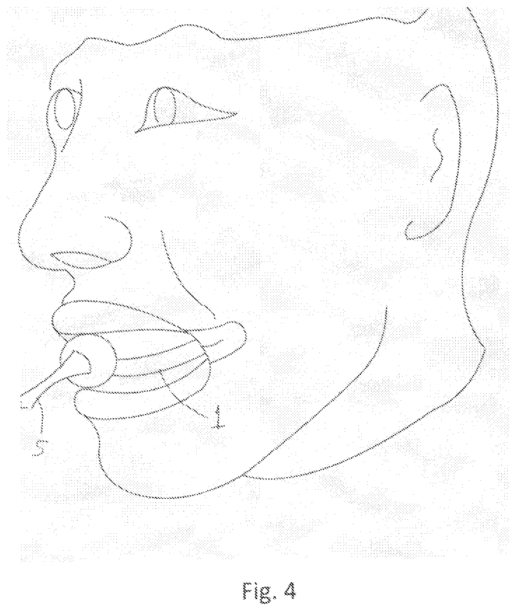

As shown in FIGS. 3 and 5, the opposing channels 10 of the mouthpiece 1 receive cleaning agent 9 (dentifrice tooth gel or toothpaste). The range of dentifrices toothpaste/toothgels 9 is part of the cleaning system. Each dentifrice is formulated to enhance a specific health care benefit. The dentifrices toothpaste/tooth-gels 9 contain ingredients to i) increase the formation of micro-bubbles (cavitation process), ii) decrease the surface tension of the paste, and iii) reduce the viscosity of the fluid for a better fluid adherence to the mouthpiece 1. Ingredients such as, but not limited to: biodegradable, polymer based microspheres, which enhance the possibility of cavitation, baking soda, surfactant containing materials will be used. All dentifrices 9 are formulated for best ultrasound transmission (max efficiency) and creation of nano-cleaning bubbles (cavitation effect). The dentifrices do not contain abrasive cleaning (polishing) particles as used in ordinary toothpastes formulations. The dentifrice range includes regular cleaning (mint flavor), natural cleaning (no fluoride, with herbs), gum health therapy (e.g. with oregano extract and other antibacterial ingredients), and whitening (bleaching ingredients).

To operate the mouthpiece 1—the mouthpiece 1 is wetted and connected to controller 2. The dentifrice gel 9 is selected and applied to the mouthpiece 1. The mouthpiece 1 is then inserted into mouth and a cleaning or health care mode is selected and initiated. The user then waits for acoustic and/or sensory signal to indicate the completion of the cleaning/treatment session and the controller 2 automatically turns off the mouthpiece 1. The user takes out mouthpiece 1 and rinses it in water and rinses mouth out with water. The entire regular cleaning/treatment session can be completed in 30 seconds or less. To charge and store—place the controller 3 into the charging base for inductive charging.

The mouthpiece 1 provides reduced cleaning time. Reduced time to clean teeth and gums as all surfaces are treated simultaneously and evenly. Longer cleaning for every surface—average cleaning time per each treated surface increased compared to recommended ADA (American Dental Association, Chicago Ill.) cleaning time recommendation (i.e. 2 minutes all teeth to 30 seconds for each single tooth). The mouthpiece 1 provides equally long and even cleaning performance for every dental (teeth/gum) surface and interdental spaces. This new cleaning device and method is absolutely brush technique independent and thus more repeatable. The mouthpiece 1 provides for more gentle cleaning without abrasion risk—no more abrasive scrubbing of teeth and gums. Cleaning with soft ultrasound (LIPUS) works without manual or electronic powered brushing action and without the use of abrasive cleaning (polishing) materials. This eliminates the risk of teeth and/or gum damage through brushing (no abrasive brushing and eliminates risk of wrong manual brushing technique), thus being superior to all ordinary dental cleaning brushing technologies. The mouthpiece provides more thorough cleaning—because ultrasound waves and nano-sized cleaning bubbles (cavitation effect) penetrate gums and interdental spaces where much thicker toothbrush bristles cannot reach. The mouthpiece 1 has an antibacterial effect through combination of blue UV light, Ultrasonic cavitation and/or emitted Ultrasound waves (inside mouth and mouthpiece). Additionally, this combination penetrates tissue for therapeutic effects. The mouthpiece 1 provides hands-free operation. The mouthpiece has a simpler cleaning operation—remove mouthpiece from mouth, rinse device in running water and rinse inside of mouth and done.

The effect of ultrasound in oral hygiene scientific research has proven to destroy the outer shell of bacteria and germs and does not cause damage to the human body at the indicated transmission frequencies and ultrasound power levels. The gums are gently massaged, inflammation subsides and gum regenerates again. The ultrasound penetrates and cleans in smallest crevices, in particular the ultrasound penetrates gum tissue up to 12 mm. The use of UV light in oral care is known to kill bacteria in the 470-490 mm wavelength range.

1. A mouthpiece cleaning device comprising:
a U-shaped mouthpiece skeleton defining opposing channels for receiving a user's teeth therein, said channels each having a respective teeth facing surface;
transducers disposed along said channels at said respective teeth facing surfaces;
UV LEDs disposed along said channels at said respective teeth facing services, said UV LEDs being interspersed with said transducers.

2. The mouthpiece cleaning device according to claim 1, wherein said transducers are PMUTS or CMUTS or Piezo chips.

3. The mouthpiece cleaning device according to claim 1, further comprising a sonic vibration chip disposed in said skeleton.

4. The mouthpiece cleaning device according to claim 1, wherein said transducers and said LEDs are embedded in said skeleton by injection molding.

5. The mouthpiece cleaning device according to claim 4, wherein said mouthpiece is a solid or semi-solid polymer, or gel-based material, being PVC, polyethylene, PEEK, polycarbonate, polyetherimide, polysulfone, polypropylene or polyurethane.

6. The mouthpiece cleaning device according to claim 1, further comprising a flexible circuit board, said transducers and said LEDs being disposed on said flexible circuit board.

7. The mouthpiece cleaning device according to claim 6, further comprising a controller connected to said flexible circuit board, said controller for controlling said transducers and said LEDs.

8. The mouthpiece cleaning device according to claim 1, further comprising an overmolded layer of material softer than a material of said skeleton.

9. The mouthpiece cleaning device according to claim 8, further comprising spacers disposed in said channels at said respective teeth facing surface, said spacers for contacting the user's teeth and maintaining a minimum distance between said transducers and the teeth, said spacers being formed of said overmolded layer.
